(19) United States
(12) Patent Application Publication
(10) Pub. No.: US 2008/0131534 A1
(43) Pub. Date: Jun. 5, 2008

(54) USE OF A PLANT EXTRACT OR PLANT JUICE

(75) Inventors: Alois JUNGBAUER, Vienna (AT); Monika MULLER, Wieselburg (AT)

Correspondence Address: FULBRIGHT & JAWORSKI L.L.P., 600 CONGRESS AVE., SUITE 2400, AUSTIN, TX, 78701, US

(73) Assignee: Melbrosin International Produktions- und Vertriebs GesmbH & Co KG, Vienna (AT)

(21) Appl. No.: 11/940,786

(22) Filed: Nov. 15, 2007

(30) Foreign Application Priority Data
Nov. 15, 2006 (EP) 06450165.3

(51) Int. Cl. A61K 36/53; A61K 36/8962; A61K 36/87; A61K 36/00; A61P 3/00; A61P 9/00

(52) U.S. Cl. 424/745; 424/754; 424/766; 424/725

(57) ABSTRACT

The present invention provides methods for increasing peroxisome proliferator-activated receptor-gamma (PPARγ) activity and/or endothelial nitric oxide synthase (eNOS) activity in a subject by administering to the subject a plant extract or plant juice from thyme, oregano, clove, nutmeg, red clover, bay leaves, red onion or grapes.

### BACKGROUND OF THE INVENTION

A. Field of the Invention

The present invention relates to the field of plants and plant extracts with medical or pharmaceutical use.

B. Description of Related Art

Metabolic syndrome is a combination of medical disorders that affect a large number of people in a clustered fashion. The end result of which is to increase one's risk for cardiovascular disease and diabetes. In most cases metabolic syndrome culminates in type 2 diabetes. The symptoms of metabolic syndrome are related to lipid and carbohydrate metabolism and include obesity, elevated triglycerides, low levels of high density lipoproteins, increased blood pressure or hypertension and increased glucose levels, but also symptoms of inflammation (Grundy, 2006). Generally, the individual symptoms associated with the metabolic syndrome are treated separately (e.g. diuretics and ACE inhibitors for hypertension, statins to decrease cholesterol levels or glitazones to treat diabetes). The problem is compounded when multiple drugs are necessary to control multiple risk factors. For example, once type 2 diabetes develops in patients with the metabolic syndrome, patients often require 10 or more drugs for treatment.

A new and potentially efficacious class of drugs for the metabolic syndrome as a whole, as well as for patients with type 2 diabetes, is the thiazolidinediones (TZDs). These drugs act by agonizing the nuclear receptor PPAR-gamma, which is predominantly expressed in adipose tissue, but also occurs in other tissues (Grundy, 2006; WO 2005/027661; CA 2 526 589). TZDs reduce the secretion of unesterified fatty acids and adipokines such as tumour-necrosis factor-alpha (TNF-alpha), other inflammatory cytokines, resistin and plasminogen-activator inhibitor 1 (PAI1); they also enhance adipose-tissue release of adiponectin. The net result of these changes, apparently, is to reduce insulin resistance in muscle and liver and to mitigate prothrombotic and pro-inflammatory states. These findings therefore suggest that TZDs are hitting at the heart of the metabolic syndrome by improving insulin resistance in adipose tissue. Still, in type 2 diabetes, they have only a modest effect on plasma lipoproteins and blood pressure. So, although they improve the metabolic syndrome, they by no means cure it once type 2 diabetes develops.

The PPAR-gamma is a class II nuclear receptor that forms a heterodimer with the retinoid X-receptor (RXR) and binds to specific regions on the DNA of target genes. Expression of target genes is increased or decreased, depending on the gene. PPARγ is composed of 6 structural regions in 4 functional domains. The A/B region forms the ligand-independent transactivation domain which can be covalently modified by phosphorylation. By the C region, the DNA-binding domain of the receptor can be targeted to the PPARγresponse element (PPRE), a specific sequence of nucleotides within the regulatory region of responsive genes. The E/F region contains the ligand binding domain and the co-activator/co-repressor-binding surface. Binding of agonists leads to conformational changes of the receptor and to its activation. The activated receptor heterodimerizes with RXR and this heterodimer binds to PPRE through the DNA-binding domain (Guo, 2006).

Natural ligands of the PPAR-gamma receptor are fatty acids such as lauric acid, petroselenic acid, linolenic acid, linoleic acid and arachidonic acid, fatty acid metabolites like 15-deoxy-delta 12,14-prostaglandin J2. The synthetic ligands comprise the group of thiazolidinedions (Troglitazone, Rosiglitazone, Pioglitazone), the non-thiazolidinediones (e.g. GW1929, GW7845) and the non-steroidal anti-inflammatory drugs (e.g. flufenamic acid, fenoprofen).

PPAR-gamma downregulates TNF-alpha, leptin, IL-6, plasminogen activator inhibitor-1 (PAI-1), resistin, 11-beta-hydroxysteroid dehydrogenase type-1 (11-beta-HSD-1). Those proteins are responsible for insulin resistance. Thus activation of PPAR-gamma leads to reduced insulin resistance because they regulate the glucose-transporter protein GLUT-4 in the cell membrane.

Insulin resistance is a key factor in development of the metabolic syndrome. This syndrome is the coexistence of hyperglycaemia, hypertension, dyslipidemia and obesity. Therefore cardiovascular diseases such as coronary heart diseases and stroke are more prevalent among patients with metabolic syndrome (Gurnell et al., 2003).

WO 2005/053724 mentions non-aqueous extracts of Astragalus membranaceus which influence PPAR-gamma. Compounds found in these extracts are for example calycosin, formononetin, genistein, afromorsin, biochanin A, coumestrol, odoratin and daidzein. PPAR-gamma mediated diseases which can be treated with the extracts are dyslipidaemia, atherosclerosis, coronary heart disease, obesity and colon cancer.

WO 2005/027661 describes catechines, which are obtainable from green tea, as activating ligands of PPAR-gamma. The ligands mentioned therein, in particular glitazone, rosiglitazone, ciglitazone and pioglitazone, fall under the group of TZDs. Further TZDs, for example troglitazone are mentioned in the US 2006/0030597 A1.

CA 2 526 589 A1 describes ligands of PPAR-gamma, in particular glabrene, glabridine, glabrol and their derivatives, and glitazones. These compounds are mentioned in connection with the multiple risk factor syndrome, another name of the metabolic syndrome, which is related to insulin resistance and can be treated with PPAR-gamma ligands. Also described is a licorice extract for the treatment of metabolic syndrome.

EP 1 350 516 B1 claims a hydrophobic licorice extract, and extracts from turmeric, clove and cinnamon for the use of treating metabolic syndrome as well as associated diseases like visceral obesity and diabetes mellitus. The activity of the extracts is measured in reference to troglitazone and pioglitazone.

JP 2005/097216 mentions dehydrodieugenol A and B, magnolol, oleanic acid and betulic acid as PPAR-gamma ligands that are useful for preventing or ameliorating metabolic syndrome.

Kwon, Young-In I et al. (Asia Pacific Journal of Clinical Nutrition 15(1) (2006):107-118) describe plants that have inhibitory action on the α-amylase, α-glycosidase and ACE and therefore are considered for a treatment of diabetes and increased blood pressure.

McCue, Patrick et al. (Asia Pacific Journal of Clinical Nutrition 13(4) (2004):401-408) also describe the efficacy of extracts of oregano and specific compounds, e.g. rosmarinic acid and Quercetin on the activity of α-amylase through the inhibition of the enzyme. Symptoms like hyperglycaemia, type 2 diabetes and prediabetes impaired glucose tolerance could be treated.

Lemhadri, A et al. (Journal of Ethnopharmacology 92(2-3) (2004):251-256) show the anti-hyperglycemic effects of oregano extracts, which were exemplified on a type 1 diabetes mellitus model (streptozotocin-diabetic rats).

Grover, J K et al. (Journal of Ethnopharmacology 81(1) (2002):81-100) describe traditional Indian plants with anti-diabetic effect. Among these plants are onions with anti-hyperglycemic activity.

Ajay, Machha et al. (Diabetes Research and Clinical Practice 73(1) (2006):1-7) show improved protection of vascular vessels of Quercetin by enhanced endothelial nitric oxide bioavailability.

### SUMMARY OF THE INVENTION

It is the goal of the present invention to provide a pharmaceutical preparation with particular exceptional potential to threat or prevent metabolic syndrome and diseases associated with metabolic syndrome.

Therefore the present invention provides the use of a plant extract or plant juice for the production of a pharmaceutical or nutritional preparation for the treatment or prevention of metabolic syndrome, wherein the plant extract or juice is from thyme, oregano, clove, nutmeg, red clover, bay leaves, red onion or grapes. The extracts activate the receptor PPAR-gamma and thus induce a reduction of blood glucose. For example the extracts can be made with DMSO (dimethylsulfoxide) as extracting agent. The final extract or juice may have other solvents, preferably nutritional or pharmaceutically suitable solvents. It was now surprisingly found that preparations of extracts or juices of these plants can be used to treat metabolic syndrome. The term juice includes fermented juices or beverages such as wines, especially red wine made from grape vines, e.g. Vitis sp.

In one embodiment, the present invention provides a method of increasing peroxisome proliferator-activated receptor-gamma (PPARγ) activity in a subject comprising administering to a subject a plant extract or plant juice from thyme, oregano, clove, nutmeg, red clover, bay leaves, red onion or grapes, wherein the PPARγactivity is increased.

In another embodiment, the present invention provides a method of increasing peroxisome proliferator-activated receptor-gamma (PPARγ) activity in a subject comprising: obtaining a composition comprising an isolated compound of one or more of Quercetin, 2-Hydroxychalcone, Luteolin, Cinnamaldehyde, Diosmetin, 2′-Hydroxychalcone, Phloretin, Isoquercetrin, Myricetin, Equol, Eriodictyol, ODMA (O-Desmethyl-angolenain), Resveratrol, Catechin, Apigenin, Vitexin, Gallic acid, Galangin, Naringin, Eugenol, Eriodictyol, Apigenin, Taxifolin, Salvianolic acid B, Chrysoeriol, Kaempferol, Thymol, Carvacrol, Safrol, Ethylcinnamate, Limonene or a mixture thereof, and administering the composition to the subject, wherein the PPARγactivity is increased.

In one embodiment, the present invention provides a method of increasing endothelial nitric oxide synthase (eNOS) activity in a subject comprising administering to a subject a plant extract or plant juice from thyme, oregano, clove, nutmeg, red clover, bay leaves, red onion or grapes, wherein the eNOS activity is increased.

In another embodiment, the present invention provides a method of increasing endothelial nitric oxide synthase (eNOS) activity in a subject comprising: obtaining a composition comprising an isolated compound of one or more of Quercetin, 2-Hydroxychalcone, Luteolin, Cinnamaldehyde, Diosmetin, 2′-Hydroxychalcone, Phloretin, Isoquercetrin, Myricetin, Equol, Eriodictyol, ODMA (O-Desmethyl-angolenain), Resveratrol, Catechin, Apigenin, Vitexin, Gallic acid, Galangin, Naringin, Eugenol, Eriodictyol, Apigenin, Taxifolin, Salvianolic acid B, Chrysoeriol, Kaempferol, Thymol, Carvacrol, Safrol, Ethylcinnamate, Limonene or a mixture thereof, and administering the composition to a subject, wherein the eNOS activity is increased.

In certain embodiments, the present invention provides a method of treating or preventing metabolic syndrome in a subject comprising administering to a subject a plant extract or juice from thyme, oregano, clove, nutmeg, red clover, bay leaves, red onion or grapes, wherein the metabolic syndrome is treated or prevented.

In another embodiment, the present invention provides a method of treating or preventing metabolic syndrome in a subject comprising: obtaining a composition comprising an isolated compound of one or more of Quercetin, 2-Hydroxychalcone, Luteolin, Cinnamaldehyde, Diosmetin, 2′-Hydroxychalcone, Phloretin, Isoquercetrin, Myricetin, Equol, Eriodictyol, ODMA (O-Desmethyl-angolenain), Resveratrol, Catechin, Apigenin, Vitexin, Gallic acid, Galangin, Naringin, Eugenol, Eriodictyol, Apigenin, Taxifolin, Salvianolic acid B, Chrysoeriol, Kaempferol, Thymol, Carvacrol, Safrol, Ethylcinnamate, Limonene or a mixture thereof, and administering the composition to a subject, wherein the metabolic syndrome is treated or prevented.

In certain aspects of the invention the plant extract, plant juice, or isolated compounds have an EC50-value of PPAR-gamma binding of less than 50 μg/ml, less than 35 μg/ml, less than 20 μg/ml, less than 10 μg/ml, less than 5 μg/ml, or less than 1 μg/ml based on the dry-weight of the plant.

In some aspects of the invention the plant extract, plant juice, or isolated compounds have an eNOS activity of between about 3 pMol/mg to 40 pMol/mg, 4 to 35 pMol/mg, 5 pMol/mg to 30 pMol/mg, or 6 pMol/mg to 25 pMol/mg.

In certain aspects of the invention the isolated compound or mixture of isolated compounds of Quercetin, 2-Hydroxychalcone, Luteolin, Cinnamaldehyde, Diosmetin, 2′-Hydroxychalcone, Phloretin, Isoquercetrin, Myricetin, Equol, Eriodictyol, ODMA (O-Desmethyl-angolenain), Resveratrol, Catechin, Apigenin, Vitexin, Gallic acid, Galangin, Naringin, Eugenol, Eriodictyol, Apigenin, Taxifolin, Salvianolic acid B, Chrysoeriol, Kaempferol, Thymol, Carvacrol, Safrol, Ethylcinnamate, or Limonene in the composition has an EC50-value of PPAR-gamma binding of less than 900 μM, less than 500 μM, less than 150 μM, less than 70 μg/ml, less than 55 μM, or less than 15 μM. In some aspects the isolated compound or mixture of isolated compounds in the composition has an eNOS activity of about 3 pMol/mg to 40 pMol/mg, 4 to 35 pMol/mg, 5 pMol/mg to 30 pMol/mg, or 6 pMol/mg to 25 pMol/mg.

In certain embodiments, the subject has metabolic syndrome. In some embodiments, the metabolic syndrome may be associated with a lack of activation of PPAR-gamma. In some embodiments, the subject has one or more of the following conditions: obesity, dyslipidaemia, hypolipidaemia, insulin resistance, and/or arteriosclerosis. In certain aspects of the invention, the subject has a circulatory disorder, cardiovascular disease, or stenosis. The circulatory disorder, cardiovascular disease, or stenosis may or may not be in combination with metabolic syndrome. In certain aspects of the invention, the subject does not have diabetes. In certain aspects, the subject has a fasting plasma glucose level of less than 126 mg/dL. In some aspects, the subject has a fasting plasma glucose level of between 110-125 mg/dL. In certain aspects of the invention, the methods further comprise assessing the subject's PPAR-gamma and/or eNOS activity level before treatment, after treatment, or both before and after treatment.

The plant extracts, plant juices, and compounds may be provided in a variety of forms. In certain embodiments, they are provided as a pharmaceutical preparation or a nutritional preparation. In some aspects of the invention, they are formulated as an oral preparation. The oral preparation may be, for example, a liquid, a tablet, or a capsule.

It is contemplated that any method or composition described herein can be implemented with respect to any other method or composition described herein.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Logistic dose response curves the 6 most potent plant extracts, determined by Polar Screen PPAR Competitive Assay.

FIG. 2: Logistic dose response curves of the 6 most potent unconcentrated plant extracts, determined by Polar Screen PPAR Competitive Assay.

FIG. 3: Logistic dose response curves of further plant extracts, determined by Polar Screen PPAR Competitive Assay.

FIG. 4: Logistic dose response curves of isolated compounds of different plant extracts, determined by Polar Screen PPAR Competitive Assay.

FIG. 5: eNOS activity of plant extracts.

FIG. 6: Results of eNOS assay.

FIG. 7: Results of nitrite assay.

### DESCRIPTION OF ILLUSTRATIVE EMBODIMENTS

Rare mutations in the human PPAR-gamma are associated with lower transactivational capacity or complete loss of function lead to development of a certain type of familial lipodystrophy in the bearers, associated with all signs of metabolic syndrome. The PPAR-gamma agonist rosiglitazone has been shown to improve insulin resistance in such cases. Also genetically modified animals with loss of function of the PPAR-gamma exhibit features of the metabolic syndrome.

The metabolic syndrome, also termed “insulin resistance syndrome” is a non-diabetic accumulation of risk factors, which can lead to the development of diabetes but it is not identical with diabetes. As defined by the American Association of Clinical Endocrinology the metabolic syndrome (i.e. the Insulin Resistance Syndrome) is defined by four factors:

Insulin Resistance Syndrome, i.e. Metabolic Syndrome

| | | | |
|---|---|---|---|
| 1. | Triglycerides | >150 | mg/dL |
| 2. | HDL cholesterol | | |
| | Men | <40 | mg/dL |
| | Women | <50 | mg/dL |
| 3. | Blood pressure | >130/85 | mm Hg |
| 4. | Glucose | | |
| | Fasting | 110-125 | mg/dL |
| | 120 min post-glucose challenge | 140-200 | mg/dL |

### (ACE Position Statement, Endocr Pract. 9(3), 2003: 240-252)

In comparison to this definition the World Health Organization (WHO) defines diabetes as the following:

Diabetes mellitus

| | Venous plasma glucose concentration | |
|---|---|---|
| | mmol/l | mg/dL |
| fasting or | ≧7.0 | ≧126 |
| 2-hour post-75 g glucose load | ≧11.1 | ≧200 |

### (Khatib, Guidelines for the prevention, management and care of diabetes mellitus, EMRO Technical Publications Series 32, chapter 2 p. 13-19; 2006).

The abnormalities related to elevated triglycerides, HDL cholesterol values and increased blood pressure are characteristics of the metabolic syndrome. Drugs for the treatment of metabolic syndrome are required to enhance insulin sensitivity, as well as the other manifestations of the insulin resistance syndrome. This action was surprisingly found in the plants and compounds of the pre-sent invention which interact with the PPAR-gamma. PPAR-gamma interaction has a beneficial effect on insulin sensitivity but also on the lipid profile, blood pressure, haemostasis and can significantly reduce the risk of cardiovascular disease associated with metabolic syndrome (Walcher et al., Diabetes and Vascular Res. 2004 (2): 76-81).

In contrast, impaired transactivation, which has been described in the frequent genetic polymorphism Ala12Pro of the PPAR-gamma gene, leads to higher insulin sensitivity (but lower postprandial hypertriglyceridemia), and a haplotype for which higher transcriptional activity is postulated has an increased risk for metabolic syndrome. Therefore, a non-linear activity-effect curve has been proposed for PPAR-gamma activity, with small increases in activity having opposite effects than stronger activation by ligands.

A lot of plants and plant extracts have hypoglycemic activity. Most prominent representatives of these plants/plant extracts are: Cinnamon, cinnamon powder/cinnamon bark powder (Cinnamomum verum), bitter melon (Momordica charantia), cumin (Cuminum cyminum), tumeric (Curcuma longa) and fenugreek (Trigonella foenum-graecum). These extracts are currently used or marketed for treatment of diabetes II. Other plant extracts have been mentioned to have hypoglycemic activities. These are: clove (Syzygium aromaticum), oregano (Origanum sp.), thyme (Thymus vulgaris), nutmeg (Myristica fragrans), alfalfa (Medicago sativa), red clover (Trifolium pratense), bay leaves (Laurus nobilis), white cabbage (Brassica oleracea var. capitata f. alba), curly hale (Brassica oleracea convar. acephala var. sabellica) and black pepper (Piper nigrum). In all above mentioned cases the activation of Peroxisome proliferator-activated receptor gamma was unknown.

Also disclosed herein is the method of treating a patient with metabolic syndrome or preventing metabolic syndrome with a preparation described herein. “Preventing” or “prevention” herein does not require absolute success in the sense of an absolute prevention but indicates a reduced risk of developing metabolic syndrome.

Preferably the plant extract or juice is from oregano. Oregano has been attributed anti-hyperglycemic activity (McCue, 2004; Yaniv, 1987; Lemhadri, 2004; McCue, 2004). It has been hypothesised that oregano extracts and rosmaric acid as a compound present in oregano inhibit pancreatic amylase and thus exhibit anti-hyperglycemic activity. Inhibition of starch break down to glucose contributes to the management of hyperglycemia and diabetes complication in the long term. It has been also mentioned that oregano inhibits aldose reductase, the first enzyme of the polyol pathway implicated in the secondary complications of diabetes (Koukoulitsa, 2006). However the indications were never strong enough to promote an activity against metabolic syndrome. The compounds in oregano have been thoroughly examined (see table 3 below). However it was found that the individual activity of the constituents in isolated form can only explain about 10% of the PPAR-gamma activity of oregano plant extracts. This takes the extreme divergence of oregano batches into account (see table 4). The PPAR-gamma activity of oregano strongly varies depending on growth factors like soil composition, light intake, and cutting time—these factors seem to be more important than the choice of oregano species and strain (see table 2).

Preferably the plant extract or juice has an EC₅₀-value of the PPAR-gamma binding of less than 50 μg/ml, preferably less than 35 μg/ml, more preferred less than 20 μg/ml, even more preferred less than 10 μg/ml, especially preferred less than 5 μg/ml, most preferred less than 1 μg/ml based on the dry-weight of the plant. These values can be determined by the assay described in the examples section and influenced as mentioned above by the selection of an appropriate batch in the case of strong activity variance as in oregano.

In another aspect of the present invention the use of isolated compounds selected from Quercetin, 2-Hydroxychalcone, Luteolin, Cinnamaldehyde, Diosmetin, 2′-Hydroxychalcone, Phloretin, Isoquercetrin, Myricetin, Equol, Eriodictyol, ODMA (O-Desmethyl-angolenain), Resveratrol, Catechin, Apigenin, Vitexin, Gallic acid, Galangin, Naringin, Eugenol, Eriodictyol, Apigenin, Taxifolin, Salvianolic acid B, Chrysoeriol, Kaempferol, Thymol, Carvacrol, Safrol, Ethylcinnamate, Limonene or mixtures thereof, preferably selected from Quercetin, Luteolin, Diosmetin, Isoquercetrin, Eridictoyl, Naringenin, Eriodictyol, Apigenin, Vitexin, Taxifolin, Salvianolic acid B, Chrysoeriol, Kaempferol, Thymol, Carvacrol or mixtures thereof, is provided for the production of a pharmaceutical or nutritional preparation for the treatment or prevention of metabolic syndrome. Especially preferred are those compounds found in oregano given in table 3 below or those from table 1. These compounds have been tested and could now for the first time be attributed with adequate PPAR-gamma binding activity which enables a use against metabolic syndrome. These compounds can be provided as a pharmaceutical or nutritional preparation for oral intake, for example as juice or as tablet. Additionally the plant extracts or juices can be selected to comprise one or more of these substances in a further embodiment.

In preferred embodiments the compounds of the preparation have an EC₅₀-value of the PPAR-gamma binding of less than 900 μM, preferably less than 500 μM, more preferred less than 150 μM, even more preferred less than 70 μM, especially preferred less than 55 μM, most preferred less than 15 μM. Adequate compounds can be selected alone or in combination, for example given the information of table 1 below.

All preparations (e.g. of the extract or of the compounds) described so far can be used to treat, ameliorate or prevent metabolic syndrome. Preferably the metabolic syndrome is associated with insulin resistance and/or a lack of activation of PPAR-gamma. Activity of PPAR-gamma can be determined in vitro with a sample with the Peroxisome Proliferator-Activated Receptor-gamma Competitor Assay Kit, Green (Invitrogen) as exemplified below. Stimulation of the PPAR-gamma with the extract, juice or active compound can circumvent malign effects of a lack of PPAR-gamma activation.

In further embodiments the metabolic syndrome is associated with diabetes, obesity, dyslipidaemia, hypolipidaemia, insulin resistance or arteriosclerosis. Cardiovascular diseases are a consequence of metabolic syndrome and can also be an associated indicator of metabolic syndrome. Symptoms of these diseases normally occur in the development of metabolic syndrome and can be treated with the inventive preparations. In particular the preparations are effective to treat the symptoms of diabetes type 2 since the PPAR-gamma activation can ameliorate glucose sensitivity. Therefore in a preferred embodiment the metabolic syndrome is associated with the clinical symptoms of diabetes type 2.

Additionally the present invention provides for a use of as described herein, wherein the extract, juice or compounds are used for the production of a pharmaceutical or nutritional preparation for the treatment of metabolic syndrome and/or at least one of selected from circulatory disorders and stenosis. It was surprisingly found that the preparations of the present invention can also be used for this combination treatment. Especially the oregano extracts increase the activity of eNOS (Endothelial Nitric Oxide Synthase). eNOS generates nitric oxide in blood vessels and is involved with regulating vascular function. eNOS is associated with plasma membranes surrounding cells and the membranes of Golgi bodies within cells. Nitric oxide is synthesized from arginine and oxygen. In the endothelium of blood vessels nitric oxide is the signal to the surrounding smooth muscle to relax, thus dilating the artery and increasing blood flow. This additional effects of the preparations allows the treatment of both metabolic syndrome and circulatory disorders (independently or in combination) with only one medicament. Therefore one aspect of the present invention is the use of preparation (of the extracts, juices or the active compounds) for increasing the activity of eNOS in a patient (human or animal) and for the treatment of eNOS associated diseases like circulatory disorders, cardiovascular disease and stenosis (also in combination with metabolic syndrome).

Preferably the extract, juice or compounds have an eNOS activity of 3 pMol/mg to 40 pMol/mg, preferably of 4 to 35 pMol/mg, even more preferred of 5 pMol/mg to 30 pMol/mg, most preferred of 6 pMol/mg to 25 pMol/mg. The eNos activity can be determined by the methods described below and can be selected by varying the constituents and their concentration in their preparation.

In a preferred embodiment, the pharmaceutical preparation according to the invention comprises a pharmaceutical carrier. A pharmaceutical carrier includes wetting, emulsifying, or pH buffering agents or vehicles with which the pharmaceutical preparation can be contained in or administered. Examples are oils, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Preferably, the agents of the pharmaceutical preparation are formulated as salt forms. Pharmaceutically acceptable salts include those formed with anions such as those derived from hydrochloric, phosphoric, acetic, oxalic, tartaric, butyric acids, etc., and those formed with cations such as those derived from sodium, potassium, ammonium, calcium, ferric hydroxides, isopropylamine, triethylamine, 2-ethylamino ethanol, histidine, procaine, etc.

Preferably the preparation is an oral preparation, in particular preferred in form of a juice or tablet. The extracts, juices or compounds can be dried and formulated into tablets and administered orally. For example, the preparations can be formulated in the form of tablets, capsules, cachets, gelcaps, solutions, suspensions, and the like. Tablets or capsules can be prepared by conventional means with pharmaceutically acceptable excipients such as binding agents (e.g., pregelatinized maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may take the form of, but are not limited to, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-phydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

The following examples are included to demonstrate certain embodiments of the invention. Those of skill in the art should, in light of the present disclosure, appreciate that many changes can be made in the specific embodiments that are disclosed and still obtain a like or similar result without departing from the spirit and scope of the invention.

### Example 1

### PPAR-Gamma Assay and PPAR-Gamma Binding Activity

1.1 Extraction and Preparation of the Plants/Plant Powders

100 mg dry powder of plants, herbs or spices was suspended in 1 ml DMSO for 24 h at room temperature. The suspension was stirred on a magnetic stirrer. After 24 h the extract was clarified by centrifugation for 1 h at 13.000 rpm. The clear supernatant was further diluted by DMSO and then binding to PPARγwas tested by Peroxisome Proliferator-Activated Receptor-gamma Competitor Assay Kit, Green (Invitrogen, Carlsbad, Calif.).

1.2 Polar Screen PPAR Competitive Assay

PPAR-gamma Competitive assay was performed according to manufacturer's instructions as described in the PolarScreen™ PPAR Competitor Assay, Green Protocol (839-0412498 060904). PPAR-gamma-LBD and the fluorescent PPAR-gamma ligand (Fluormone™ PPAR Green) form a PPAR-gamma-LBD/Fluormone™ PPAR-gamma Green complex which has a high polarization value. If a competitor of PPAR-gamma is added the fluorescent ligand is displaced. This causes that the Fluoromone is free in solution and tumbles rapidly during its fluorescence lifetime which results in a low polarisation value. Substances which do not compete will not displace the fluorescent ligand from the complex so that the high polarisation value remains.

1 μl of test compounds, which was dissolved and diluted in DMSO, was transferred into a microwell plate. Complete screening buffer was prepared by addition of 0.5 μl DTT (1M) to 1 ml PPAR Green Screening Buffer. 19 μl of this buffer was added to each well of microwell plate. The PPAR-LBD/Fluoromone Green complex solution was prepared by addition of 16 μl Fluoromone PPAR Green solution (500 nM) per 1.6 ml and 2 μl PPAR-gamma-Ligand binding domain (1.56 mg/ml) per 1.6 ml to complete screening buffer. 20 μl of this solution was added to each well. The plate is covered to protect the reagents from light and incubated for two hours at room temperature. Fluorescence polarization was measured at excitation wavelength 485 nm and emission 535 nm with Tecan Genios Pro plate reader (Tecan, Austria).

1.3 Curve Fitting

Data of the ligand binding assay were calculated in the following way: Polarization values were plotted against the concentration of test compound. The curve was fitted using a logistic dose-response model from Table Curve 2D software (Jandel Scientific). The used function was:

$$y = a + \frac{b}{1 + {( c / x )}^{d}}$$

The parameter a equals the baseline, b is the difference between the plateau of the curve and the baseline and c is the transition center of the curve which is the concentration that causes 50% of efficiency (ligand potency). d gives the transition zone and is a measure for positive or negative co-operatively.

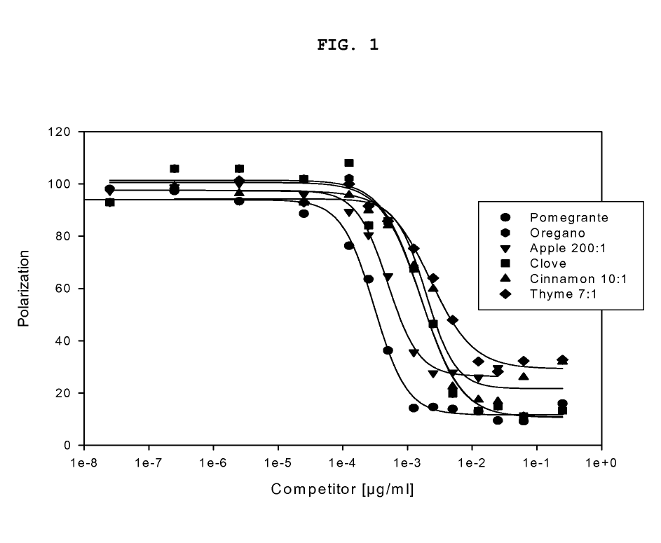

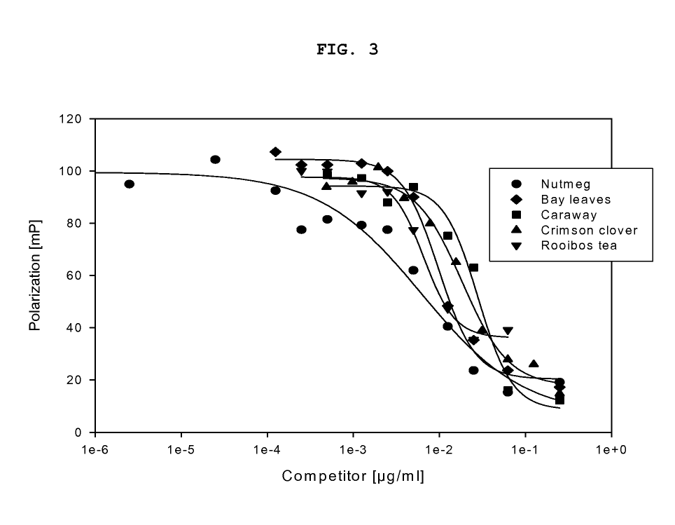

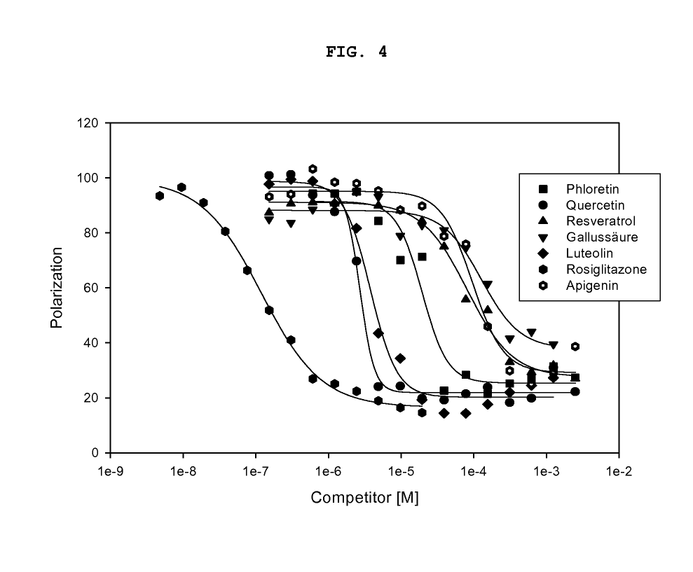

The concentration of the test compound that results in a half-maximal shift in polarization value is the EC₅₀ value of the test compound. This value serves as measure of relative binding affinity to PPAR-gamma. Fitted curves are depicted in FIGS. 1 to 4.

TABLE 1
List of tested substances:

| Substance | EC₅₀ | |
|---|---|---|
| CW9662 (2-Chloro-5-nitrobenanilide) | 10.7 | nM |
| Rosiglitazone | 120 | nM |
| Pioglitazone | 253 | nM |
| Troglitazone | 1.8 | μM |
| Quercetin | 2.6 | μM |
| 2-Hydroxychalcone | 2.9 | μM |
| Luteolin | 4.0 | μM |
| Rosmarinic acid | 7.8 | μM |
| Coumestrol | 8.5 | μM |
| Cinnamaldehyde | 10.4 | μM |
| Diosmetin | 13.7 | μM |
| Biochanin A | 20 | μM |
| 2′-Hydroxychalcone | 20 | μM |
| Phloretin | 20 | μM |
| Genistein | 22 | μM |
| Ciglitazone | 23 | μM |
| Isoquercetrin | 52 | μM |
| Myricetin | 53 | μM |
| Equol | 60 | μM |
| Eriodictyol | 66 | μM |
| ODMA (O-Desmethyl-angolenain) | 67 | μM |
| Resveratrol | 81 | μM |
| Catechin | 85 | μM |
| Apigenin | 104 | μM |
| Gallic acid | 128 | μM |
| Galangin | 143 | μM |
| Naringin | 257 | μM |
| Eugenol | 420 | μM |
| Kaempferol | 467 | μM |
| Daidzein | 470 | μM |
| Thymol | 695 | μM |
| Carvacrol | 723 | μM |
| Safrol | 2.25 | mM |
| Ethylcinnamate | 2.70 | mM |
| Limonene | 9.59 | mM |
| Phloridzin | >500 | μM |
| Epicatechin | >500 | μM |
| Enterodiol | >500 | μM |
| Chrysin | >500 | μM |
| 3-Hydroxyflavon | >500 | μM |
| Enterolacton | >500 | μM |
| Hesperidin | >500 | μM |
| Capsaicin | >500 | μM |
| Indol-3-Carbinol | >500 | μM |
| p-Coumaric acid | >500 | μM |

| | |
|---|---|
| Hyperoside | does not bind |
| Curcumin | does not bind |
| Sesamin | does not bind |
| Diosmin | does not bind* |
| Tamoxifen | does not bind* |
| Caffeic acid | does not bind* |
| Coumestan | does not bind* |
| Procyanidin B2 | does not bind* |
| Prunetin | does not bind* |
| Daidzin | does not bind* |
| Genistin | does not bind* |
| Ononin | does not bind* |
| Sissotrin | does not bind* |
| Puerarin | does not bind* |
| Formononetin | does not bind* |
| ICI | does not bind* |
| o-Coumaric acid | does not bind* |
| Chlorogenic acid | does not bind* |
| Estron | does not bind* |
| Protocatechuic acid | does not bind* |
| Linalool | does not bind* |
| β-Caryophyllene | does not bind* |
| Ocimene | does not bind* |
| p-Cymene | does not bind* |
| 2-Heptanon | does not bind* |
| Alliin | does not bind* |
| Diallylsulfide | does not bind* |
| 2′-OH-Chalcone | does not bind |
| Ferrulic acid | does not bind |
| α-Humulene | does not bind* |

*tested to a concentration of 0.1 M

TABLE 2
Plant extracts

| Plant, concentrated extract | | | |
|---|---|---|---|
| fold | supplier | EC₅₀ | |
| pomegranate (fruit) | Styrka Botanics | 307 | ng/ml |
| 40% ellagic acid | | | |
| apple 200:1 | Pfannnenschmidt | 467 | ng/ml |
| 5% Quercetin, 30% Phloridzin | | | |
| clove | Kotany | 0.85 | μg/ml |
| oregano vulgare ssp. vulgare | Kotany | 1.6 | μg/ml |
| oregano vulgare ssp. vulgare | Spar | 1.8 | μg/ml |
| cinnamon 10:1 | Eurochem | 1.94 | μg/ml |
| thyme 7:1 | EuroChem | 2.26 | μg/ml |
| Oregano vulgare | McCormick | 3.21 | μg/ml |
| cinnamon | Kotany | 3.75 | μg/ml |
| green coffee | Exxentia | 4.11 | μg/ml |
| Trigonella | Calendula | 4.49 | μg/ml |
| blueberry, 35% phenole, | Pharmalink | 4.55 | μg/ml |
| 10% anthocyanine | | | |
| thyme | Kotany | 6.10 | μg/ml |
| nutmeg | Kotany | 6.28 | μg/ml |
| rooibos tea | | 6.35 | μg/ml |
| red clover blossom extract 20% | | 7.46 | μg/ml |
| bay leaves | Kotany | 9.81 | μg/ml |
| oregano blossom | | 10.93 | μg/ml |
| red clover extract 40% | | 12.88 | μg/ml |
| Origanum vulgare vulgare | Galke | 15.13 | μg/ml |
| (Oregani cretici) | | | |
| tulsi 5:1, tannins | Pfannenschmidt | 16.10 | μg/ml |
| tulsi 5:1, 1% ursol acid | Pfannenschmidt | 16.37 | μg/ml |
| blue berry 5:1 | Pharmalink | 17.35 | μg/ml |
| crimson clover blossoms | Südburgenland | 18.44 | μg/ml |
| holy basil | Galke | 25.34 | μg/ml |
| oregano 20:1 | Exxentia | 28.00 | μg/ml |
| caraway | Kotany | 28.23 | μg/ml |
| oregano vulgare | Fuchs | 28.55 | μg/ml |
| stevia tea | | 28.78 | μg/ml |
| cacao | Bensdorf | 32.36 | μg/ml |
| oregano 15:1 | Exxentia | 32.75 | μg/ml |
| Mexican oregano | | 39.32 | μg/ml |
| marjoram | Kotany | 39.80 | μg/ml |
| tulsi | Galke | 40.18 | μg/ml |
| oregano vulgare (greenware) | Exxentia | 43.21 | μg/ml |
| Matai fungi | | 53.75 | μg/ml |
| Helichrysum italicum (curry | garden | 54.92 | μg/ml |
| plant) | (Mühlviertel) | | |
| rosemary | Kotany | 54.58 | μg/ml |
| tarragon | garden (Mühlviertel) | 56.86 | |
| rosemary 2,4% rosemary acid | Exxentia | 56.95 | μg/ml |
| ginseng | Exxentia | 57.71 | μg/ml |
| kale extract | NCI | 65.65 | μg/ml |
| oregano vulgare | Hamburger Gewürz- | 65.73 | μg/ml |
| | mühle | | |
| Turkish oregano türkisch | Paul Bruns | 70.23 | μg/ml |
| black pepper | Kotany | 71.02 | μg/ml |
| menoflavone | Melbrosin | 79.53 | μg/ml |
| onion, red | NCI | 84.00 | μg/ml |
| oregano 15:1 | Naturex | 95.53 | μg/ml |
| santolina chamaecyarissus | garden | 100.04 | μg/ml |
| | (Mühlviertel) | | |
| oregano vulgare | garden | 100.11 | μg/ml |
| | (Mühlviertel) | | |
| thyme | Fuchs | 109.29 | μg/ml |
| borage blossom | garden | 114.5 | μg/ml |
| | (Mühlviertel) | | |
| fucus | | 116.6 | μg/ml |
| alfalfa | Pfannenschmidt | 137 | μg/ml |
| dill | Kotany | 197 | μg/ml |
| nasturtium | garden | 223.7 | μg/ml |
| | (Mühlviertel) | | |
| Jiaogulan tea | | 241.01 | μg/ml |
| white cabbage extract | NCI | 289 | μg/ml |
| Mulberry tea | | 290.26 | μg/ml |
| marigold blossom | garden | 323.91 | μg/ml |
| | (Mühlviertel) | | |
| oregano 4:1 | Pfannenschmidt | 388 | μg/ml |
| alfalfa sprouts | health food shop | 437 | μg/ml |
| lucerne | pet shop | 486 | μg/ml |
| (Grünmehl-Extrakt) | | | |
| opuntia | Pfannenschmidt | ~960 | μg/ml |
| sauerkraut juice | Ja natürlich | >500 | μg/ml |
| oregano 5:1 | Exxentia | >500 | μg/ml |
| wood garlic | | >500 | μg/ml |
| currant | Styrka Botanics | >500 | μg/ml |
| lovage | garden | >500 | μg/ml |
| | (Mühlviertel) | | |
| trigonella | Styrka Botanics | >500 | μg/ml |
| trigonella | Fenulife | >500 | μg/ml |
| ginseng | anhui | >500 | μg/ml |
| phaseolus vulgaris | Linnea | >500 | μg/ml |
| chive | NCI | >500 | μg/ml |
| parsley | NCI | >500 | μg/ml |
| leaves of | Südburgenland | >500 | μg/ml |
| santolina chamaecyarissus | | | |
| lavender blossom | | >500 | μg/ml |

| | |
|---|---|
| Kudzu | does not bind |
| cress | does not bind |
| kvass | does not bind |
| celeriac must | does not bind |

TABLE 3
Active substances in Oregano

| Substance | EC₅₀ | |
|---|---|---|
| Quercetin | 2.6 | μM |
| Luteolin | 4.0 | μM |
| Rosmarinic acid | 7.8 | μM |
| Diosmetin | 13 | μM |
| Biochanin A | 20 | μM |
| Isoquercetrin | 53 | μM |
| Eriodictyol | 66 | μM |
| Narigenin | 81 | μM |
| Apigenin | 104 | μM |
| Vitexin | 156 | μM |
| Naringin | 257 | μM |
| Taxifolin | 275 | μM |
| Salvianolic acid B | 292 | μM |
| Chrysoeriol | 323 | μM |
| Kaempferol | 467 | μM |
| Thymol | 695 | μM |
| Carvacrol | 723 | μM |
| Limonene | 9.59 | mM |
| PeonidinCl | >500 | μM |
| Ursolsäure | >500 | μM |
| Apigenin-7Glycoside | >500 | μM |
| p-Coumaric acid | >500 | μM |
| Vanillic acid | >500 | μM |

TABLE 4
PPAR-γ binding assays of different
oregano batches (supplier: Kotany)

| extract | EC₅₀ | |
|---|---|---|
| Oregano 1 | 1.6 | μg/ml |
| Oregano 2 | 1.8 | μg/ml |
| Oregano 3 | 3.4 | μg/ml |
| Oregano 4 | 34 | μg/ml |
| Oregano 5 | 35 | μg/ml |
| Oregano 6 | 41 | μg/ml |

### Example 2

### PPAR-Gamma Activation of Wines

The wines described in table 5 have been tested as described in example 1.

TABLE 5
EC₅₀ values of PPAR-gamma ligand binding assays
of selected red wines. It was assumed that each
wine contains 20 g/l dry substance.

| | EC₅₀: | | EC₅0: | |
|---|---|---|---|---|
| | without concen- | | Normalised for 20 | |
| | tration, filtered | | g/l dry substance | |
| Chianti classico, Italy | 707 | nl/ml | 14 | μg/ml |
| Valpolicella, Italy | 440 | nl/ml | 8.8 | μg/ml |
| Malbec, Argentina | 436 | μl/ml | 8.7 | μg/ml |
| Bordeaux, France | 22 | μl/ml | 435 | μg/ml |
| Zinfandel, California | 44 | μ/ml | 888 | μg/ml |
| Zweigelt Reserve, Austria | 87 | μl/ml | 1.7 | mg/ml |
| Zweigelt classic, Austria | 120 | μl/ml | 2.4 | mg/ml |
| Cabernet sauvignon, Chile | 34 | μl/ml | 682 | μg/ml |

### Example 3

### eNOS Activation

Cell Cultures and Treatments

Human umbilical vein endothelial cells (HUVECs) were harvested enzymatically with type I A collagenase (1 mg/ml) as previously described (Simoncini et al., 1999) and maintained in phenol red-free DMEM, containing HEPES (25 mM), heparin (50 U/ml), endothelial cell growth factor (50 ng/ml), L-glutamine (2 mM), antibiotics, and 10% fetal bovine serum (FBS). Before each experiment, HUVECs were kept for at least 48 h in DMEM containing 10% steroid-deprived FBS. All experiments were performed on confluent monolayers of endothelial cells. Before every experiment investigating rapid, nontranscriptional effects (up to 30-min treatments), HUVECs were serum starved in DMEM containing no FBS for 8 h before treatment to avoid the confounding effects of serum. Inhibitor were added 30 min before the treatments.

eNOS Activity Assay

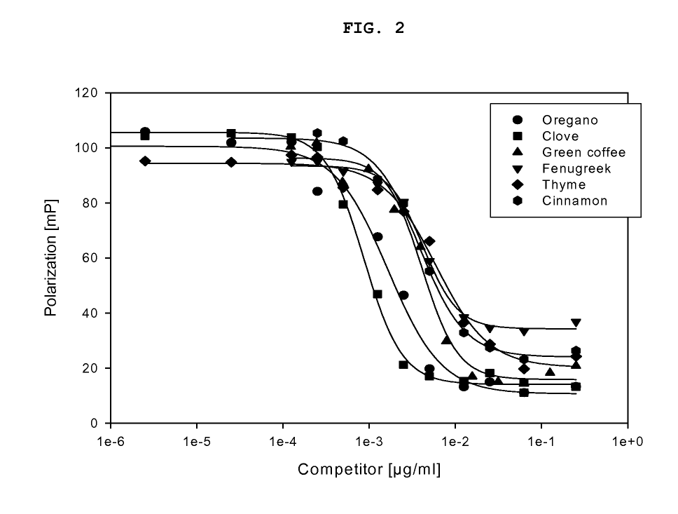

eNOS activity was determined as conversion of [³H]arginine to [3H]citrullinein endothelial cell lysates. Converted citrulline was separated by unconverted arginine using the acidic ion-exchange resin Dowex 50 W, 200-400, as described (Simoncini et al., 2000). Extracts incubated with the eNOS inhibitor, N-nitro-L-arginine methyl ester (1 mM), served as blank (FIG. 2).

### Nitrite Assay

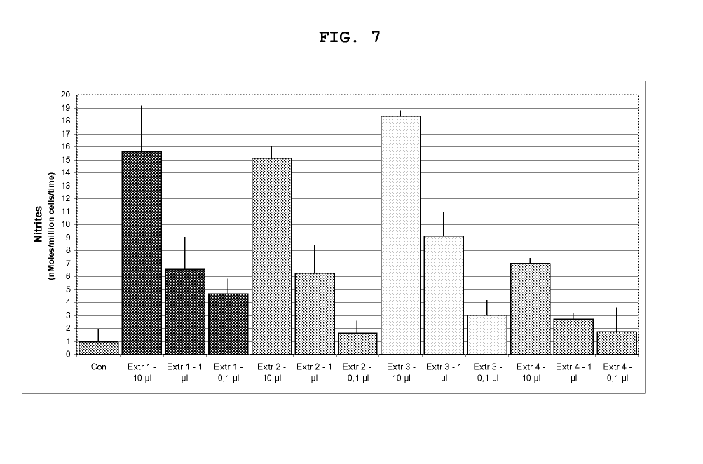

Nitric oxide production was determined by a modified nitrite assay using 2,3-diaminonaphtalene as described (Simoncini and Genazzani, 2000). Fluorescence of 1-(H)naphtotriazole was measured with excitation and emission wavelengths of 365 and 450 nm. Standard curves were constructed with sodium nitrite (FIG. 7). Nonspecific fluorescence was determined in the presence of NG-monomethyl-L-arginine (3 mM).

Extract 1: Diazimt

Extract 2: Thyme 7:1

Extract 3: Oregano (Kotany)

Extract 4: cinnamon (Kotany)

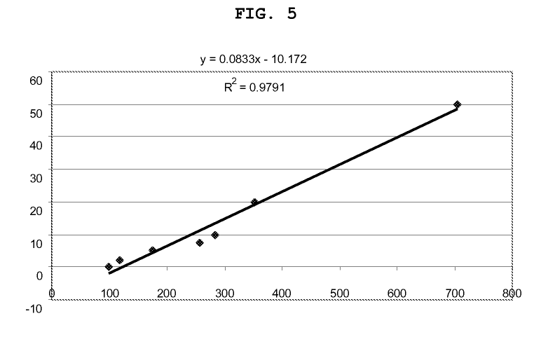

TABLE 6
Values of the nitrite standard curve as illustrated in FIG. 5.

| Nitrites | | | |
|---|---|---|---|
| Concentration | extracts dose dependency 48 hours | | |
| in μM | | Nitrite (standard curve) | mean |

| | | | |
|---|---|---|---|
| 0 | 100 | 99 | 99.5 |
| 2 | 123 | 114 | 118.5 |
| 5 | 177 | 172 | 174.5 |
| 7.5 | 250 | 263 | 256.5 |
| 10 | 277 | 290 | 283.5 |
| 20 | 338 | 367 | 352.5 |
| 50 | 688 | 720 | 704 |

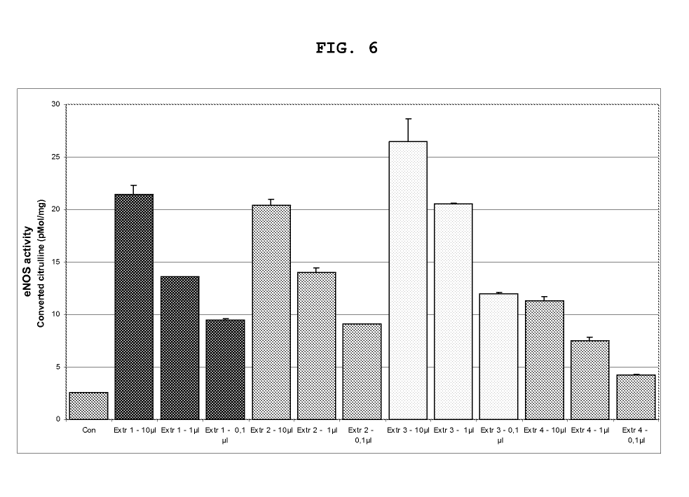

TABLE 7
modifying effects of plant extracts on the
activity of eNOS - illustrated in FIG. 6:

| | eNOS activity |
|---|---|
| Extract - Concentration | pMol/mg |
| blank | 2.561628 |
| Extr 1 - 10 μl | 21.4162 |
| Extr 1 - 1 μl | 13.56301 |
| Extr 1 - 0.1 μl | 9.481734 |
| Extr 2 - 10 μl | 20.3668 |
| Extr 2 - 1 μl | 14.01594 |
| Extr 2 - 0.1 μl | 9.056034 |
| Extr 3 - 10 μl | 26.45035 |
| Extr 3 - 1 μl | 20.50787 |
| Extr 3 - 0.1 μl | 11.94931 |
| Extr 4 - 10 μl | 11.27611 |
| Extr 4 - 1 μl | 7.501733 |
| Extr 4 - 0.1 μl | 4.242154 |

eNOS Results

The effect of oregano extract, thyme extract and two different cinnamon extracts on NO synthesis and endothelial nitric oxide synthase (eNOS) activity in human endothelial cell was tested (FIG. 6). Different amounts of plant extracts (100 mg/ml DMSO) were administered to serum-starved, steroid-deprived human umbilical vein endothelial cells (HUVECs) and incubated for 48 hours. Exposure to oregano extract resulted in a concentration-dependent increase in NO release in the cell culture medium which was associated with a parallel activation of eNOS.

In comparison to thyme and cinnamon extract oregano had a higher influence on eNOS activation. 1 μl of oregano extract (100 mg/ml) had higher activation potential as 10 μl thyme or 10 μl cinnamon extract (100 mg/ml).

All of the compositions and methods disclosed and claimed herein can be made and executed without undue experimentation in light of the present disclosure. While the compositions and methods of this invention have been described in terms of certain embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

Grundy, Nat. Rev. Drug Disc. 5 (2006): 295-309
Gurnell et al., Journal of Clinical Endocrinology and Metabolism (88) (2003): 2412-2421
Koukoulitsa et al., Bioorganic and Medicinal Chemistry (14) (2006): 1653-1659
Lemhadri et al., Journal of Ethnopharmacology (92) (2004): 251-256
Guo et al., Pharmacology and Therapeutics 111(1) (2006): 145-73
McCue et al., Asia Pacific Journal of Clinical Nutrition (13) (2004): 401-408
McCue et al., Asia Pacific Journal of Clinical Nutrition (13) (2004): 101-106
Yaniv et al., Journal of Ethnopharmacology (19) (1987): 145-151
Simoncini et al. Journal of Clinical Endocrinology and Metabolism 84 (1999): 2802-2806.
Simoncini and Genazzani. Journal of Clinical Endocrinology and Metabolism 85 (2000): 2966-2969.
Simoncini et al. Nature 407 (2000): 538-541.

1. A method of treating or preventing metabolic syndrome in a subject comprising administering to a subject a plant extract or plant juice from thyme, oregano, clove, nutmeg, red clover, bay leaves, red onion or grapes, wherein the metabolic syndrome is treated or prevented.

2. The method of claim 1, wherein the plant extract or plant juice is from oregano.

3. The method of claim 1, wherein the plant extract or plant juice has an EC₅₀-value of PPAR-gamma binding of less than 50 μg/ml based on the dry-weight of the plant.

4. The method of claim 1, wherein the plant extract or plant juice has an EC₅₀-value of PPAR-gamma binding of less than 1 μg/ml based on the dry-weight of the plant.

5. The method of claim 1, wherein the metabolic syndrome is associated with a lack of activation of peroxisome proliferator-activated receptor-gamma (PPARγ).

6. The method of claim 1, wherein the plant extract or plant juice is formulated as a tablet or capsule.

7. A method of treating or preventing metabolic syndrome in a subject comprising:
(a) obtaining a composition comprising an isolated compound of one or more of Quercetin, 2-Hydroxychalcone, Luteolin, Cinnamaldehyde, Diosmetin, 2′-Hydroxychalcone, Phloretin, Isoquercetrin, Myricetin, Equol, Eriodictyol, ODMA (O-Desmethyl-angolenain), Resveratrol, Catechin, Apigenin, Vitexin, Gallic acid, Galangin, Naringin, Eugenol, Eriodictyol, Apigenin, Taxifolin, Salvianolic acid B, Chrysoeriol, Kaempferol, Thymol, Carvacrol, Safrol, Ethylcinnamate, Limonene or a mixture thereof; and
(b) administering the composition to a subject, wherein the metabolic syndrome is treated or prevented.

8. The method of claim 7, wherein the composition comprises an isolated compound of one or more of Quercetin, Luteolin, Diosmetin, Isoquercetrin, Eridictoyl, Naringenin, Eriodictyol, Apigenin, Vitexin, Taxifolin, Salvianolic acid B, Chrysoeriol, Kaempferol, Thymol, Carvacrol or a mixture thereof.

9. The method of claim 7, wherein the composition is a pharmaceutical preparation.

10. The method of claim 7, wherein the composition is a nutritional preparation.

11. The method of claim 7, wherein the compound or mixture of compounds in the composition has an EC₅₀-value of PPAR-gamma binding of less than 900 μM.

12. The method of claim 7, wherein the compound or mixture of compounds in the composition has an EC₅₀-value of PPAR-gamma binding of less than 15 μM.

13. The method of claim 7, wherein the metabolic syndrome is associated with a lack of activation of peroxisome proliferator-activated receptor-gamma (PPARγ).

14. The method of claim 7, wherein the composition is formulated as an oral preparation.

15. The method of claim 14, wherein the oral preparation is a liquid.

16. The method of claim 14, wherein the oral preparation is a tablet or capsule.

17. A method of increasing endothelial nitric oxide synthase (eNOS) activity in a subject comprising administering to a subject a plant extract or plant juice from thyme, oregano, clove, nutmeg, red clover, bay leaves, red onion or grapes, wherein the eNOS activity is increased.

18. The method of claim 17, wherein the plant extract or plant juice is from oregano.

19. The method of claim 17, wherein the subject has cardiovascular disease.

20. The method of claim 17, wherein the subject has stenosis.

21. The method of claim 17, wherein the plant extract or plant juice has an eNOS activity of 3 pMol/mg to 40 pMol/mg.

22. The method of claim 17, wherein the plant extract or plant juice has an eNOS activity of 6 pMol/mg to 25 pMol/mg.

23. The method of claim 17, wherein the plant extract or plant juice is formulated as a tablet or capsule.

24. A method of increasing endothelial nitric oxide synthase (eNOS) activity in a subject comprising:
(a) obtaining a composition comprising an isolated compound of one or more of Quercetin, 2-Hydroxychalcone, Luteolin, Cinnamaldehyde, Diosmetin, 2′-Hydroxychalcone, Phloretin, Isoquercetrin, Myricetin, Equol, Eriodictyol, ODMA (O-Desmethyl-angolenain), Resveratrol, Catechin, Apigenin, Vitexin, Gallic acid, Galangin, Naringin, Eugenol, Eriodictyol, Apigenin, Taxifolin, Salvianolic acid B, Chrysoeriol, Kaempferol, Thymol, Carvacrol, Safrol, Ethylcinnamate, Limonene or a mixture thereof, and
(b) administering the composition to a subject, wherein the eNOS activity is increased.

25. The method of claim 24, wherein the composition comprises an isolated compound of one or more of Quercetin, Luteolin, Diosmetin, Isoquercetrin, Eridictoyl, Naringenin, Eriodictyol, Apigenin, Vitexin, Taxifolin, Salvianolic acid B, Chrysoeriol, Kaempferol, Thymol, Carvacrol or a mixture thereof.

26. The method of claim 24, wherein the composition is a pharmaceutical preparation.

27. The method of claim 24, wherein the composition is a nutritional preparation.

28. The method of claim 24, wherein the subject has cardiovascular disease.

29. The method of claim 24, wherein the subject has stenosis.

30. The method of claim 24, wherein the compound or mixture of compounds in the composition has an eNOS activity of 3 pMol/mg to 40 pMol/mg.

31. The method of claim 24, wherein the compound or mixture of compounds in the composition has an eNOS activity of 6 pMol/mg to 25 pMol/mg.

32. The method of claim 24, wherein the composition is formulated as an oral preparation.

33. The method of claim 32, wherein the oral preparation is a liquid.

34. The method of claim 32, wherein the oral preparation is a tablet or capsule.
